(19) United States
(12) Patent Application Publication
(10) Pub. No.: US 2025/0197951 A1
(43) Pub. Date: Jun. 19, 2025

(54) METHODS OF ISOLATING SALMONELLA SEROTYPES

(71) Applicant: UNIVERSITY OF GEORGIA RESEARCH FOUNDATION, INC., Athens, GA (US)

(72) Inventor: Nusrat SHARIAT, Watkinsville, GA (US)

(21) Appl. No.: 18/849,612

(22) PCT Filed: Mar. 24, 2023
(86) PCT No.: PCT/US2023/016274

§ 371 Date: Sep. 23, 2024

Related U.S. Application Data
(60) Provisional application No. 63/323,472, filed on Mar. 24, 2022.

(51) Int. Cl. C12Q 1/689; C12Q 1/24; C12Q 1/6874

(52) U.S. Cl. CPC C12Q 1/689; C12Q 1/24; C12Q 1/6874; G01N 2333/255

(57) ABSTRACT

This disclosure relates to methods of isolating rare or non-dominant serotypes of Salmonella species from a sample. The disclosure further relates to the isolation of non-dominant serotypes of Salmonella in a sample related to one or more products for human or animal consumption.

### CROSS REFERENCE TO RELATED APPLICATIONS

The present application claims priority to and the benefit of U.S. Provisional Patent Application. No. 63/323,472, filed Mar. 24, 2022, which is incorporated by reference in its entirety for all purposes.

### FIELD

This disclosure relates to methods of isolating rare or non-dominant serotypes of Salmonella species from a sample.

### BACKGROUND

Salmonella is a genus of gram-negative bacteria which is currently classified into two species, Salmonella bongori and Salmonella enterica. S. enterica is a diverse species which infects and colonizes many animals including humans, while S. bongori is rarely associated with human infection. S. enterica is separated into over 2600 different serotypes, that are each defined by their O and H antigens on the cell surface. Traditional Salmonella serotyping relies on serum agglutination of these factors. First, the O group is defined with O serum (identifies the serogroup that the isolate belongs to), followed by the H antigens to complete the serotyping.

Salmonella is both genetically and phenotypically diverse. Differences among serotypes include illness severity (Cheng et al., 2019, Front Microbiol 10:1-20), antibiotic resistance (Shah et al. 2016, Poult Sci 96:687-702), geography (Strawn et al. 2014, Appl Environ Microbiol 80:3982-3991; Hendriksen et al. 2011, Foodborne Pathog Dis 8:887-900), plus host restriction and adaptation (Uzzau et al. 2000. Epidemiol Infect 125:229-255).

Some serotypes (e.g. S. typhimurium) are frequently linked to human illness (Tack et al. 2019, MMWR Morb Mortal Wkly Rep 68:369-373) while others are not. Serotyping information is crucial to food safety, as only a hundred or so of the 2,600+ known serotypes are associated with human illness. Approximately 30 serotypes account for the vast majority of morbidity in the US. It is becoming increasingly evident that Salmonella in food animals (e.g. poultry, cattle, swine) exists as multi-serotype populations. Current surveillance relies on Salmonella isolation and characterization of a few resulting colonies that grow on indicator agar (Andrews et al. 2021, Chapter 5: Salmonella. In Bacteriological Analytical Manual. U.S. Food and Drug Administration; U.S. Department of Agriculture-Food Safety and Inspection Service. 2017. MLG 4.03), typically 1-2 colonies. This is a serious limitation in Salmonella surveillance and source tracking as generally only the most abundant serotype(s) in a mixed population are detected, while the less abundant serotype(s) remain undetected (Cox et al. 2019, J Food Prot 82:1688-1696; Cason et al. 2011, abstr 262P. In International Poultry Scientific Forum. Southern Poultry Science Society, Mississippi State, MS). Where clinically important serotypes are undetected, traditional surveillance underestimates the presence of Salmonella serotypes of the greatest food safety concern.

Salmonella contamination of poultry products remains a significant cause of foodborne illness in the United States. Despite significant reduction of Salmonella during poultry processing, there has not been a concurrent reduction in human Salmonella cases that are linked to poultry. This demonstrates a need for additional Salmonella controls during live production/pre-harvest to reduce the Salmonella load on broilers entering the plant. Such controls include the use of autogenous vaccination, where serotypes of concern are used to generate killed vaccines to target those serotypes. To generate appropriate autogenous vaccines, isolates need to be isolated and characterized to determine a risk level of that particular strain, followed by the decision to include that strain as part of an autogenous vaccine. Where the most abundant serotype in a sample has a low association with human illness (e.g. serotype S. kentucky), this will likely be the isolate collected and screened for autogenous vaccine use. Less abundant isolates remain masked, or hidden, by this abundant serotype and may confer a greater public health risk (e.g. serotype S. infantis). While it is possible to define the population of Salmonella serotypes in individual samples using CRISPR-SeroSeq, it still remains a challenge to isolate and culture low frequency Salmonella to be able to characterize them and use them in autogenous vaccines.

### SUMMARY

In one aspect, provided herein is a method of enriching a sample for one or more non-dominant Salmonella serotypes, the method comprising: (a) obtaining a sample comprising (i) one or more dominant Salmonella serotypes; and (ii) one or more non-dominant Salmonella serotypes; (b) identifying the serotype of the one or more dominant Salmonella serotypes and of the one of more non-dominant Salmonella serotypes; (c) contacting the sample with an antibody against one of the dominant Salmonella serotypes and allowing sufficient time for the antibody to bind; (d) removing the antibody with the bound Salmonella serotype from the sample; and (e) collecting the remaining sample and identifying the serotype of the remaining Salmonella serotypes.

In some embodiments the sample is enriched for a short period of time to increase cell numbers.

In some embodiments, the antibodies aggregate the cells to be removed and the non-aggregated cells are removed.

In some embodiments, before step (e), steps (c) and (d) are repeated for each of the dominant Salmonella serotypes in the sample. In some embodiments, in step (c) the sample is contacted with an antibody against each of the dominant Salmonella serotypes simultaneously. In some embodiments, one dominant Salmonella serotype is present, which accounts for at least 80%, at least 85%, at least 90%, at least 95%, or at least 98% of serotypes in the sample. In some embodiments, one dominant Salmonella serotype is present, which accounts for at least 99% of serotypes in the sample. In some embodiments, one dominant Salmonella serotype is present, which accounts for at least 99.9% of serotypes in the sample.

In some embodiments, two or more dominant Salmonella serotypes are present, each of which accounts for at least 10%, a least 20%, at least 30%, or at least 40% of the serotypes in the sample. In some embodiments, two or more dominant Salmonella serotypes are present, which cumulatively account for at least 80%, at least 85%, at least 90%, at least 95%, or at least 98% of serotypes in the sample. In some embodiments, two or more dominant Salmonella serotypes are present, which cumulatively account for at least 99% of serotypes in the sample. In some embodiments, two or more dominant Salmonella serotypes are present, which cumulatively account for at least 99.9% of serotypes in the sample.

In some embodiments, one non-dominant Salmonella serotype is present, which accounts for less than 1% of serotypes in the sample. In some embodiments, one non-dominant Salmonella serotype is present, which accounts for less than 0.1% of serotypes in the sample. In some embodiments, one non-dominant Salmonella serotype is present, which accounts for less than 0.01% of serotypes in the sample.

In some embodiments, two or more non-dominant Salmonella serotypes are present, each of which accounts for less than 1% of serotypes in the sample. In some embodiments, two or more non-dominant Salmonella serotypes are present, each of which accounts for less than 0.1% of serotypes in the sample. In some embodiments, two or more non-dominant Salmonella serotypes are present, each of which accounts for less than 0.01% of serotypes in the sample.

In some embodiments, the dominant Salmonella serotype is not associated with human disease. In some embodiments, the non-dominant Salmonella serotype is associated with human disease.

In some embodiments, the serotype identification in step (b) and/or (e) is carried out using CRISPR-SeroSeq or metagenomics. In some embodiments, the antibody is an immune serum, a purified polyclonal antibody, a purified monoclonal antibody, an aptamer or another binder specific for the serogroup or serotype of the dominant serotypes. In some embodiments, the antibody causes agglutination of the dominant serotype(s).

In some embodiments, the antibody is attached to a bead. In some embodiments, the bead is made of latex, polymethyl methacrylate (PMMA), or polystyrene. In some embodiments, the bead is magnetic. In some embodiments, the antibody is present on a membrane or filter. In some embodiments, the antibody is present on the capture region of a microfluidic device. In some embodiments, the antibody is present on the capture region of a ferrofluidic device.

In some embodiments, the sample is taken from a product intended for human consumption. In some embodiments, the sample is taken from an animal intended for human consumption. In some embodiments, the sample is taken from the environment of an animal intended for human consumption. In some embodiments, the sample is a pre-harvest sample. In some embodiments, the sample is a post-harvest sample.

### BRIEF DESCRIPTION OF THE FIGURES

FIGS. 1A and 1B demonstrate the serotyping of a Salmonella population. FIG. 1A shows a mixed population of Salmonella in broth culture (left) and on agar (right) and demonstrates the challenge: it would be logistically unfeasible to pick several colonies to be certain that the non-dominant (triangle) colony was picked. Therefore, there is a need to enrich for the non-dominant colony by eliminating or reducing the proportion of the dominant (circular) colonies.

FIG. 1B shows the agglutination step to reduce the dominant (circular) colonies, thus increasing the ability to select the non-dominant (triangle) colony.

FIG. 2 shows the relative abundance of dominant and less abundant serotypes of Salmonella present in 12 environmental field samples from 3 commercial broiler farms in the US as determined by CRISPR-SeroSeq. In all cases, the dominant serotype is S. kentucky and the less abundant serotypes varies between samples, even those from the same farm (F refers to farm; H refers to house on the farm).

### DETAILED DESCRIPTION

Most Salmonella testing is carried out on samples comprising more than one serotype of Salmonella, but currently available methods lack the sensitivity to isolate non-dominant Salmonella serotypes. This disclosure provides methods to enrich samples comprising multiple serotypes for non-dominant serotypes so that these non-dominant serotypes can be isolated.

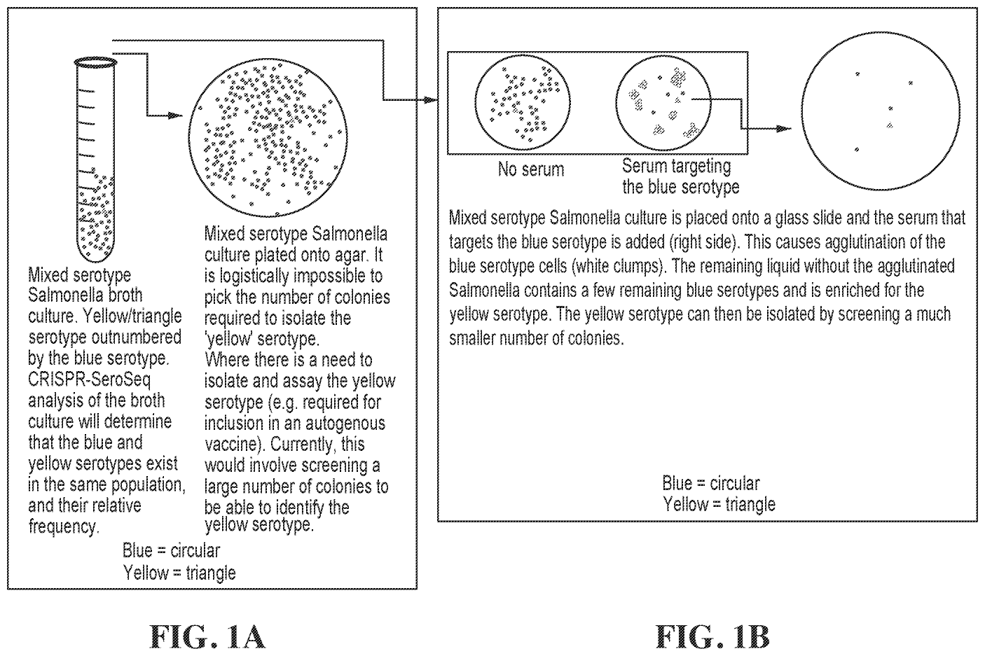

Provided herein are methods of serum exclusion that allow the enrichment of a sample for non-dominant Salmonella serotypes by removing dominant strains from the sample. Serum exclusion generally involves applying an anti-serum that contains antibodies specific to the antigen (O or H) on a dominant serotype which is present in abundance (shown as circular colonies in FIGS. 1A and 1B) to a mixed culture of Salmonella. Without wishing to be bound by theory or mechanism, it is believed that the antibodies in the anti-serum will bind to the Salmonella antigen and agglutinate with the abundant, dominant serotype, causing it to form a precipitate. The remaining liquid contains the dominant serotype in less abundance, thus enriching the sample for the non-dominant serotype. The resulting sample can then be plated on to agar and smaller number of colonies (<10) than would be required without enrichment is screened to isolate serotype B.

### Dominant and Non-Dominant Serotypes

The term “dominant serotype” in the context of this disclosure is meant a Salmonella serotype that is present at high (in some embodiments, present at the highest) relative frequency of all Salmonella serotypes in a sample. In some embodiments, a dominant Salmonella serotype accounts for about 30% to about 40%, about 40% to about 50%, about 50% to about 60%, about 60% to about 70%, about 70% to about 80%, about 80% to about 90%, or about 90% to 100% of the total number of cells of all Salmonella serotypes in a sample. In some embodiments, a dominant Salmonella serotype accounts for about 90% to about 98%, about 90% to about 95%, about 95% to about 98%, about 98% to about 99%, about 98% to 100%, or about 99% to 100% of the total number of cells of all Salmonella serotypes in a sample. In some embodiments, at dominant Salmonella serotype accounts for about 99.9% of the total number of cells of all Salmonella serotypes in a sample. In some embodiments, a dominant Salmonella serotype accounts for at least 30%, at least 40%, at least 50%, at least 60%, at least 70%, at least 80%, at least 85%, at least 90%, at least 95%, or at least 98% of the total number of cells of all salmonella serotypes in a sample. In some embodiments, a dominant Salmonella serotype accounts for more than at least 99% of the total number of cells of all Salmonella serotypes in a sample. In some embodiments, a dominant Salmonella serotype accounts for at least about 99.9% of the total number of cells of all salmonella serotypes in a sample.

A sample may contain more than one dominant Salmonella serotype. For example, two, three, four, or five dominant Salmonella serotypes may be present in a sample. If two or more dominant Salmonella serotypes are present, each of the dominant Salmonella serotypes may account for at least 30%, at least 40%, at least 50%, at least 60%, at least 70%, at least 80%, at least 85%, at least 90%, at least 95%, at least 98%, at least 99%, or at least 99.9% of the total number of cells of all Salmonella serotypes in a sample. In some embodiments, two or more Salmonella serotypes collectively account for at least 30%, at least 40%, at least 50%, at least 60%, at least 70%, at least 80%, at least 85%, at least 90%, at least 95%, at least 98%, at least 99%, or at least 99.9% of the total number of cells of all Salmonella serotypes in a sample.

As will be appreciated by a person of skill in the art, essentially any Salmonella serotype has the potential to be a dominant serotype depending on the growth conditions, the source of the sample, the host, and other factors. For example, in pre-harvest poultry, serotype S. kentucky is typically dominant and masks other serotypes (such as serotype S. infantis) that are of greater concern to human health.

The term “non-dominant” Salmonella serotype describes a Salmonella serotype that accounts for a low (in some embodiments, the lowest) frequency of all Salmonella serotypes in a sample. In some embodiments, a non-dominant Salmonella serotype accounts for about 0.1% to about 0.5%, about 0.5% to about 1%, about 1% to about 2%, about 2% to about 5%, about 5% to about 10%, about 10% to about 15%, about 15% to about 20%, about 20% to about 30%, about 30% to about 40%, or about 40% to 50% of the total number of cells of all Salmonella serotypes in a sample. In some embodiments, a non-dominant Salmonella serotype accounts for less than 50%, less than 40%, less than 30%, less than 20%, less than 10%, less than 5%, less than 1% or less than 0.1% of the total number of cells of all Salmonella serotypes in a sample.

It is often difficult to isolate non-dominant Salmonella serotypes from samples because they are often masked by the dominant serotypes and a large number of colonies needs to be screened to isolate these non-dominant serotypes. Using the enrichment methods provided herein, the non-dominant Salmonella serotypes may be enriched. In some embodiments, a non-dominant Salmonella serotype in a sample may be enriched about 2-fold, about 3-fold, about 4-fold, about 5-fold, about 6-fold, about 7-fold, about 8-fold, about 9-fold, about 10-fold, about 12-fold, about 15-fold, about 20-fold, about 30-fold, about 40-fold, about 50-fold, or about 100-fold using the methods described herein.

In some embodiments, more than one non-dominant Salmonella serotype is present in a sample. For example, two, three, four, or five non-dominant Salmonella serotypes may be present in a sample. If two or more non-dominant Salmonella serotypes are present in a sample, each of the serotypes may account for less than 50%, less than 40%, less than 30%, less than 20%, less than 10%, less than 5%, less than 1% or less than 0.1% of the total number of cells of all Salmonella serotypes in a sample.

The dominant and/or the non-dominant Salmonella serotypes in a sample may be associated with human disease. In some embodiments, the non-dominant Salmonella serotype is associated with human disease but the dominant serotype is not. In some embodiments, both the dominant and the non-dominant Salmonella serotype are associated with human disease. Human illness caused by Salmonella commonly presents within 12-48 hours of ingesting the Salmonella organism. The most commonly caused illness is gastroenteritis which presents with nausea, cramping, abdominal pain, diarrhea, fever, and vomiting. While the disease is usually mild, severe cases do occur.

The dominant and/or the non-dominant Salmonella serotypes in a sample may be associated with an animal disease. In some embodiments, the non-dominant Salmonella serotype is associated with an animal disease but the dominant serotype is not. In some embodiments, both the dominant and the non-dominant Salmonella serotype are associated with an animal disease. In some embodiments, the animal is a mammal (e.g., a dog or cat, or cattle such as a cow, etc.). In some embodiments, the animal is a bird (e.g., a chicken or other poultry).

### Serotype Identification

Using information about the various Salmonella serotypes in a sample, serum exclusion can be used to remove the more abundant Salmonella serotypes that are present, thus enriching for minority Salmonella serotypes that can then be isolated from a plate by picking a much smaller number of colonies. Any suitable method known in the art or described herein can be used to determine the serotype of Salmonella colonies before and after enrichment.

A common method of serotyping Salmonella in a sample comprises applying a sample to an agar plate, incubating the agar plate under conditions that support bacterial growth (e.g., incubation at 37° C. for about 16-24 h), and then picking the resulting colonies from the plates to determine the serotype. The colonies can then be contacted with an anti-sera to different O antigen(s) (determined by the lipopolysaccharide on the surface of the Salmonella bacterium) and if agglutination (clumping) occurs with an anti-serum to a particular O antigen, the colony is positive for that particular O antigen. Once the O antigen is determined, the process is repeated with anti-sera to various H antigens (determined by the flagella protein) in a two-step process to force phase expression for the H1 and H2 antigens. The combination of antigens indicates the Salmonella serotype. The antigenic profile of ten common Salmonella serotypes is shown in Table 1.

TABLE 1
Antigenic profiles of CDC top 10 Salmonella serotypes

| Serotype | O antigen | H1 | H2 |
|---|---|---|---|
| Enteritidis | 1, 9, 12 | g, m | — |
| Newport | 6, 8, 20 | e, h | 1, 2 |
| Typhimurium | 1, 4, [5], 12 | i | 1, 5 |
| Javiana | 1, 9, 12 | l, z28 | 1, 5 |
| I 4, [5], 12: i: — | 1, 4, [5], 12 | i | — |
| Infantis | 6, 7, 14 | r | 1, 5 |
| Muenchen | 6, 8 | d | 1, 2 |
| Montevideo | 6, 7, 14 | g, m, [p], s | [1, 2, 7] |
| Braenderup | 6, 7, 14 | e, h | e, n, z |
| Thompson | 6, 7, 14 | k | 1, 5 |

Alternatively or additionally, sequencing techniques such as metagenomics, or targeted amplicon based sequencing applications such as CRISPR-SeroSeq also allow population analyses to be performed and determines the Salmonella serotype identity. Metagenomics allows for the identification of Salmonella serotypes in mixed populations using single nucleotide polymorphisms (SNPs). See Vohra et al., Appl Environ Microbiol. 2018 Feb. 15; 84 (4): e02262-17. The CRISPR-SeroSeq method has the ability to discern as many as ten serotypes in a single sample over four logs of relative abundance. See Thompson et al., Applied and Environmental Microbiology, 2018, 84 (21): e01859-18. Evaluation of a naturally occurring environmental reservoir of Salmonella using CRISPR-SeroSeq has shown that multiple Salmonella serotypes in a single sample are common, and that pathogenic Salmonella serotypes are often obscured by an overburden on non-pathogenic Salmonella serotypes, meaning that traditional serotyping methods would frequently miss serotypes of concern. See Deaven et al., Applied and Environmental Microbiology, 2021, 87 (6): e02594-20; Siceloff A T, et al. Appl Environ Microbiol. 2022 Apr. 26; 88 (8): e0020422; and Obe, T. et al. Applied and Environmental Microbiology, 2023.

The serum exclusion methods for enriching non-dominant Salmonella serotypes described herein present an opportunity to better utilize existing serotyping methods, and to isolate non-dominant serotypes from a sample.

### Applications

In another aspect, provided herein is a method of enriching a sample for one or more non-dominant Salmonella serotypes, the method comprising (a) obtaining a sample comprising one or more dominant Salmonella serotypes and one or more non-dominant Salmonella serotypes; (b) contacting the sample with an antibody against one of the dominant Salmonella serotypes and allowing sufficient time for the antibody to bind; (c) removing the antibodies with the bound Salmonella serotype from the sample; and (d) collecting the remaining sample and identifying the serotype of the remaining Salmonella serotypes. Steps (b) and (c) may be repeated for each of the dominant Salmonella serotypes before proceeding to step (d), if more than one serotype is desired to be excluded. In some embodiments, the serotype of the one or more dominant Salmonella serotypes is identified before step (a). The serotype may be identified using any suitable method know in the art, including, for example, CRISPR-SeroSeq.

The description of a process to induce agglutination of dominant salmonella species is not meant to be limited to the use of serogroup specific immune sera. A person of skill in the art will appreciate that other methods are described in the literature that may be used to achieve the same result. For example, in situations where specific sera or antibodies that cause agglutination are not available, other methods would be required to deplete the dominant serotypes from the sample.

A person of skill in the art will appreciate that any suitable antibody or other binding protein may be used in the methods provided herein, as long as the protein binds the antigen of the Salmonella serotype to be excluded. Examples of antibodies that may be used include, but are not limited to, an immune serum, a purified polyclonal antibody, a purified monoclonal antibody, an aptamer or any other binder specific for the serogroup or serotype of the dominant serotypes. Alternatively, other proteins that specifically bind to oligosaccharides may be used to bind to an antigen expressed by the Salmonella serotype to be excluded, for example, lectins. In some embodiments, the antibody or binding protein causes agglutination of the dominant Salmonella serotype(s).

In some embodiments, the antibody again the dominant Salmonella serotype is an antibody that specifically binds to the O antigen of the dominant Salmonella serotype. In some embodiments, the antibody again the dominant Salmonella serotype is an antibody that specifically binds to the H antigen of the dominant Salmonella serotype. The choice of antibody depends on the Salmonella serotypes that are sought to be excluded and enriched. For example, if the Salmonella serotype to be excluded and the Salmonella serotype to be enriched have different O antigens (e.g., S. infantis and S. typhimurium), serum exclusion may be performed using an antibody which specifically binds to the O antigen of the dominant serotype (the serotype to be excluded). If the Salmonella serotype to be excluded and the Salmonella serotype to be enriched share an O antigen (e.g., S. montevideo and S. braenderup), serum exclusion may be performed using an antibody which specifically binds to the H antigen of the dominant Salmonella serotype (the serotype to be excluded).

If more than one dominant Salmonella serotype is to be serum-excluded from a sample, the dominant serotypes may be excluded sequentially or simultaneously. In some embodiments, sequential serum exclusion begins with the serotype of the highest frequency.

The methods of serum exclusion provided herein may be carried out using any suitable techniques. For example, the antibody may be contained in an anti-serum that is added to a sample in a 96-well plate or on a microscope slide. Alternatively, the antibody may be a purified antibody.

In some embodiments, the antibody is attached to a bead and the sample is contacted with the bead. Once the dominant serotype is bound to the antibody, the bead may then be removed from the sample, thus removing the dominant serotype from the sample and leaving behind the non-dominant serotypes which can now be identified and isolated. Any suitable bead can be used to bind an antibody targeting a dominant Salmonella serotype for use in a method described herein. Beads that may be used in the methods described herein include magnetic beads, or non-magnetic beads. Magnetic beads can easily be removed from a sample using a magnet, while non-magnetic beads may be removed using centrifugation.

Magnetic beads are generally made from iron oxides and can be coated with a suitable ligand for binding antibodies, such as Protein A/G or streptavidin. Non-magnetic beads may be made of any suitable material, including, for example, latex, polymethyl methacrylate (PMMA), or polystyrene. An antibody against a dominant Salmonella serotype for use in a method described herein may be attached to a bead via a covalent interaction or a non-covalent interaction.

In some embodiments, the antibody is attached to a filter or membrane, and the sample is passed through the filter or membrane. The antibody binds the dominant serotype, allowing the non-dominant serotypes to pass through the filter or membrane.

In some embodiments, a ferrofluidic device may be used in the methods provided herein. Ferrofluids are colloidal suspensions of magnetic particles in a carrier fluid. Their magnetic properties enable ferrofluids to be used in the manipulation of cells and other particles without the need for labels. U.S. Pat. No. 10,782,223 describes methods and microfluidic devices that may be used to manipulate cells suspended in ferrofluidic solutions. The device uses ferrofluid to focus and flow a sample across a capture region which is coated with a binding agent for binding to the analyte. The ferrofluid is then used to wash the capture region, removing any unbound material, and the analyte bound to the capture region is then analyzed. In some embodiments, the capture region is coated with an antibody against a dominant Salmonella serotype.

After the dominant Salmonella serotype has been removed from a sample, the remaining sample may be plated on agar plates and cultured to produce colonies. The resulting colonies may be picked and further cultured to produce a culture of a non-dominant serotype of Salmonella. These cultures may then be used in any suitable application, including, for example, the development of vaccine candidates, assessing antibiotic resistance in a mixed population of Salmonella serotypes, or discovering new low-abundance Salmonella serotypes. In some embodiments, the cultures may be bio banked for future use.

In one aspect, the methods provided herein allow for the serotype to be isolated from the other serotypes present in a sample and used for further testing and studies, and to create, for example, vaccine candidates.

In one aspect, the methods provided herein allow for enhanced serotype mapping and epidemiological investigations with the ability to test individual isolates in a mixed sample by genotyping, antimicrobial susceptibility testing, whole genome sequencing, and/or other methods.

In another aspect, the methods provided herein have the capability to allow isolates with specific characteristics to be biobanked. Biobanked samples can then be used for further investigations, or for the development of therapeutic products such as autogenous vaccine candidates covering required specificities, prebiotics, and/or probiotics.

In another aspect, the methods provided herein may aid in the discovery of low abundance cryptic Salmonella isolates to document additional Salmonella biodiversity, and/or to characterize potential food safety threats in newly emerging serotypes/existing serotype variants.

The methods provided herein may be applied to test products and animals intended for human consumption for the presence of non-dominant Salmonella serotypes, in particular those associated with human disease, and isolate these serotypes. Outbreaks of Salmonella infections are commonly caused by ingesting food contaminated with Salmonella, especially meat, poultry, raw milk, and eggs. Disease may also be caused by direct contact with Salmonella infected animals, or by consumption of contaminated water. The methods provided herein are particularly useful for testing food, products, animals, or other samples that are suspected of containing one or more non-dominant Salmonella serotype that is associated with human disease and isolating the same.

In some embodiments, the methods provided herein are used to enrich one or more non-dominant Salmonella serotypes in a sample taken from a product intended for human consumption. In some embodiments, the methods provided herein are used to enrich one or more non-dominant Salmonella serotypes in a sample taken from an animal intended for human consumption, for example, poultry. In some embodiments, the methods provided herein are used to enrich one or more non-dominant Salmonella serotypes in a sample taken from the environment of an animal intended for human consumption, for example, from a broiler house. The sample for enrichment in accordance with the methods described herein may be obtained at any suitable point in the production of, for example, a food product intended for human consumption. For example, the sample may be a pre-harvest sample or a post-harvest sample.

### EXAMPLES

The Example(s) in this section are provided for illustration only and are not intended to limit any invention disclosed in this application.

### Example 1: Proof of Concept of Rare Serotype Recovery of Salmonella enterica Subsp. enterica in Mixed Cultures

This example describes an exemplary method for recovery of rare Salmonella enterica isolates. The following illustrative protocol may be used to demonstrate enrichment of non-dominant serotypes from a culture comprising multiple Salmonella serotypes:

### Day 1

1. Inoculate 5 ml LB broth with an isolated colony and incubate at 37° C. for 20-24 h. Do this for each strain being tested.

### Day 2

2. Dilute cultures 1:10 (0.5 ml culture+4.5 ml LB broth) and measure the OD value, then dilute cultures to an OD₆₀₀ of 0.25 (2 ml total).
3. Mix the diluted cultures (example: 90:10, 900 μl SE (O:9) to 100 μl SI (O:7)) and centrifuge at 10,000 rpm for 180 secs. Discard supernatant and resuspend the pellet in 40 μl LB broth.
4. Using a glass plate, drop 20 μl of O:9 serum (SE) and next to it, a 20 μl drop of 1×PBS as control.
5. Add 5 μl of the mixed culture (containing S. infantis and S. enteritidis or S. infantis and S. kentucky) to both serum and also to the PBS droplet.
6. Use a pipette tip to carefully mix the serum and bacterial cultures and allow agglutination to occur (flocculation should occur within 30 seconds). Leave for up to three minutes. In parallel, do the same with the PBS control.
7. After agglutination, suck up the serum/bacteria mixture and the PBS/bacteria mixture.
8. Centrifuge at 10,000 rpm for 30 secs.
9. Remove 10 μl and use for serial dilution (down to 10-5) and plate these onto LB agar and incubate at 37 C for 20-24 h.

### Day 3

10. Count colonies on plates that have 20-200 colonies on them. Pick at least 20 colonies and confirm serogroup using O:9 serum (S. enteritidis), O:7 serum (S. infantis) and O:8 serum (S. kentucky).

This protocol was used to analyze two cultures. In a first experiment, S. infantis and S. enteritidis were mixed at ratios of 50:50, 90:10 or 100:1, and S. enteritidis was serum-excluded. The percentage of colonies identified as S. infantis (“SI”) or S. enteritidis (“SE”) are shown in Table 2.

TABLE 2
Enrichment of S. Infantis by Serum Exclusion of S. Enteritidis

| Ratio | | # Of Colonies = SI (%) | | | | | | # Of Colonies = SE (%) | | | | | |
|---|---|---|---|---|---|---|---|---|---|---|---|---|---|
| SI:SE | Sample | Rep 1 | | Rep 2 | | Rep3 | | Rep 1 | | Rep 2 | | Rep3 | |
| 50:50% | O:9 Exclusion | 31 | (97%) | 32 | (100%) | 19 | (95%) | 1 | (3%) | 0 | | 1 | (5%) |
| | PBS Control | 9 | (45%) | 11 | (55%) | 10 | (50%) | 11 | (55%) | 9 | (45%) | 10 | (50%) |
| 90:10% | O:9 Exclusion | 19 | (95%) | 19 | (95%) | 17 | (85%) | 1 | (5%) | 1 | (5%) | 3 | (15%) |
| | PBS Control | 6 | (30%) | 4 | (20%) | 4 | (20%) | 14 | (70%) | 16 | (80%) | 16 | (80) |
| 100:1% | O:9 Exclusion | 2 | (10%) | 16 | (80%) | 7 | (35%) | 18 | (90%) | 4 | (20%) | 13 | (65%) |
| | PBS Control | 0 | (0%) | 0 | (0%) | 0 | (100%) | 20 | (100%) | 20 | (100%) | 20 | (100%) |

In a second experiment, S. infantis and S. kentucky were mixed at ratios of 50:50, 90:10 or 100:1, and S. kentucky was serum-excluded. The percentage of colonies identified as S. infantis (“SI”) or S. kentucky (“SK”) are shown in Table 3.

TABLE 3
Enrichment of S. Infantis by Serum Exclusion of S. Kentucky

| Ratio | | # of colonies = SI (%) | | | | | | # of colonies = SK (%) | | | | | |
|---|---|---|---|---|---|---|---|---|---|---|---|---|---|
| SI:SK | # of colonies | Rep 1 | | Rep 2 | | Rep3 | | Rep 1 | | Rep 2 | | Rep3 | |
| 50:50% | O:8 exclusion | 18 | (100%) | 19 | (95%) | 18 | (90%) | 0 | | 1 | (5%) | 2 | (10%) |
| | PBS control | 12 | (60%) | 11 | (55%) | 9 | (45%) | 8 | (40%) | 9 | (45%) | 11 | (55%) |
| 90:10% | O:8 exclusion | 8 | (40%) | 6 | (20%) | 8 | (40%) | 12 | (60%) | 14 | (80%) | 12 | (60%) |
| | PBS control | 2 | (10%) | 2 | (10%) | 2 | (10%) | 18 | (90%) | 18 | (90%) | 18 | (90%) |
| 100:1% | O:8 exclusion | 8 | (40%) | 9 | (45%) | 5 | (25%) | 12 | (60%) | 11 | (55%) | 15 | (75%) |
| | PBS control | 0 | (0%) | 0 | (0%) | 0 | (0%) | 20 | (100%) | 20 | (100%) | 20 | (100%) |

These data demonstrate that serotypes of 1% prevalence may be recovered using the enrichment methods described in this example.

### Example 2. Recovery of Rare Salmonella enterica Isolates from a Real-World Sample

This example describes an illustrative method for recovery of rare Salmonella enterica (S. enterica) isolates from a real-world sample.
1. Obtain sample: may be fecal sample, diluted boot sock sample, a poultry rinsate sample, any commonly collected sample containing Salmonella.
2. Perform Salmonella enrichment (e.g., USDA MLG) and confirm sample is Salmonella positive (alternatively; concentrate sample and screen for Salmonella).
3. Determine the sample's Salmonella profile using the CRISPR SeroSeq method to identify both the dominant and the non-dominant serotypes (as described in Thompson et al., Applied and Environmental Microbiology, 2018, 84 (21): e01859-18 and in paragraph 029 section above).
4. Identify the dominant serotype(s) and the required serogroup specific sera required.
5. Take aliquot of enrichment culture (e.g., tetrathionate broth culture) from 1-20 ml and centrifuge for five minutes at 3000 rpm. Resuspend bacterial pellet in TSB.
6. Add an aliquot of resuspended sample and mix with appropriate serum. Following aggregation, collect non-aggregated fluid (this may include centrifugation step) and plate on to agar and incubate 20-24 hr.
7. Select colonies from plate and screen to identify the enriched non-dominant serotype(s)
8. Biobank Salmonella isolates for additional analyses and applications (e.g., virulence screening, autogenous vaccine development).

### Example 3. Evaluation of Real-World Samples Collected from the Side of a Broiler House

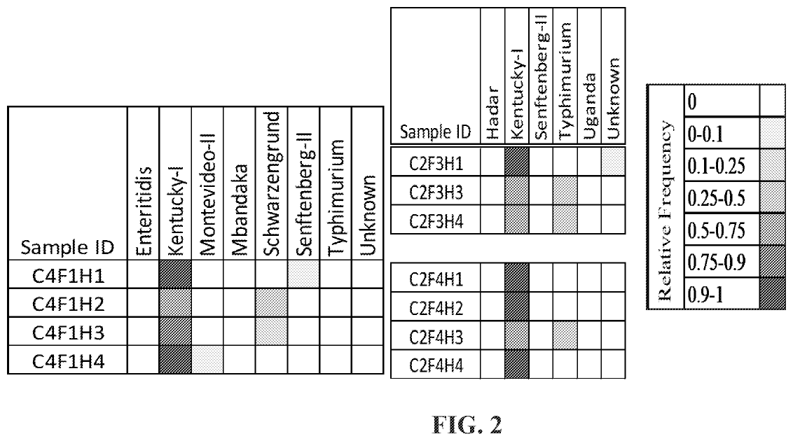

Environmental samples were collected using bootsocks moistened in buffered peptone water. A single pair of bootsocks was used to walk down the side of a broiler house to collect litter and fecal samples (results shown in FIG. 2). Broilers were 4-5 weeks old. CRISPR-SeroSeq/deep serotyping was performed on all Salmonella positive samples. Samples were cultured for Salmonella using tetrathionate selective enrichment and plated onto XLT-4. 1 ml of the selective enrichment was centrifuged and DNA isolated; the remaining 9 ml of the overnight culture was centrifuged (5 mins at 3000 rpm) and the pellet resuspended in 3 ml tryptic soy broth (TSB) containing 20% glycerol and stored at −80° C. Salmonella serotypes were profiled using CRISPR-SeroSeq deep serotyping to reveal a proportion of 64% Salmonella serotype Kentucky (S. kentucky) and 36% Salmonella serotype Schwarzengrund (S. schwarzengrund). Serum exclusion was set up to exclude serotype Kentucky (O:8).

The glycerol sample was partially thawed and four 10 ml loopfuls were transferred to 1 ml TSB and were grown for 2 h at 37° C. and 2 h at 28° C. to allow bacterial recovery. The culture was then centrifuged (5 mins at 3000 rpm) and resuspended in 30 ml of TSB. At this point, 10 ml was taken and serially diluted for the initial plate count. For the agglutination, 25 ml of O:8 serum (BD/Difco) was placed on a sterile glass plate and 5 μl of the bacterial culture added and mixed. After flocculation (agglutination) occurred, the mixture was transferred to a microcentrifuge tube and centrifuged for 10 sec up to 5000 rpm as a maximum speed to separate the flocculate from the bacteria (S. schwarzengrund) still in suspension. The supernatant was transferred to a new tube and 10-fold serial dilutions were made and plated onto XLT-4 and CASE media. As a control, 25 ml of 1×PBS was mixed with 5 ml culture and prepared in the same manner. Colonies were counted and selected from plates and then serogrouped with O:8 serum to identify S. kentucky and O:4 serum to identify S. schwarzengrund.

### Results

Based on deep serotyping, the initial proportion of S. kentucky to S. schwarzengrund was 64% to 36%. This is based on the original mixture of the two individually serotypes grown in tetrathionate (TT) broth and may not reflect the proportions when plated (i.e. if there is media bias from xylose lysine tergitol-4 (XLT-4) or chromogenic agar for Salmonella enterica (CASE)).

After serum exclusion, the proportion of the two serotypes was determined. In Replicate #1, the proportion was 67% S. schwarzengrund and 33% S. kentucky. In Replicate #2, the proportion was 15% S. schwarzengrund to 85% S. kentucky (compared to 3% S. schwarzengrund and 97% S. kentucky in the control sample with PBS). In Replicate #3, the proportion was 36% S. schwarzengrund to 64% S. kentucky (compared to 0% S. schwarzengrund and 100% S. kentucky in the control sample with PBS). In Replicate #4, the proportion was 27% S. schwarzengrund to 73% S. kentucky (compared to 13% S. schwarzengrund and 87% S. kentucky in the control sample with PBS).

| | | O:4 | O:8 | % |
|---|---|---|---|---|
| | | (Schwarzengrund) | (Kentucky) | Schwarzengrund |
| Repli- | O:8 | 4 | 2 | 67 |
| cate 1 | Exclusion | | | |
| Repli- | O:8 | 6 | 33 | 15 |
| cate 2 | Exclusion | | | |
| | PBS | 1 | 31 | 3 |
| Repli- | O:8 | 4 | 7 | 36 |
| cate 3 | Exclusion | | | |
| | PBS | 0 | 11 | 0 |
| Repli- | O:8 | 13 | 36 | 27 |
| cate 4 | Exclusion | | | |
| | PBS | 4 | 28 | 13 |

These data indicate that these methods can be used to serum-exclude dominant Salmonella strains from a real-world sample and allow for identification of a minor serotypes in the sample.

While various inventive embodiments have been described and illustrated herein, those of ordinary skill in the art will readily envision a variety of other means, steps, and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant only to be examples and that actual parameters, dimensions, materials, and configurations will depend upon the specific application or applications for which the inventive teachings is/are used. Those skilled in the art will also recognize or be able to ascertain using no more than routine experimentation, many equivalents to the specific inventive embodiments described herein. It is, therefore, to be understood that the foregoing disclosed embodiments are presented by way of example only and that, within the scope of claims supported by the present disclosure (including equivalents thereto), inventive embodiments may be practiced otherwise than as specifically described and claimed.

Some of the inventive embodiments of the present disclosure are directed to each individual feature, system, article, material, component, kit, method, and step, described herein. In addition, any combination of two or more such features, systems, articles, materials, components, kits, methods, and steps, if such features, systems, articles, materials, components, kits, methods, and steps, are not mutually inconsistent, is included within the inventive scope of the present disclosure. Some embodiments disclosed herein may also be combined with one or more features, as well as complete systems, devices or methods of other embodiments (as well as known systems, devices, or methods) to yield yet other embodiments and inventions. Moreover, some embodiments, may be distinguishable from the prior art by specifically lacking one and/or another feature disclosed in the particular prior art reference(s); i.e., claims to some embodiments may be distinguishable from the prior art by including one or more negative limitations.

Also, as shown above, various inventive concepts may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Any and all references to publications or other documents, including but not limited to, patents, patent applications, articles, webpages, books, etc., presented anywhere in the present application, are herein incorporated by reference in their entirety. Also, all references mentioned herein are specifically incorporated by reference to disclose and describe the methods and/or materials in connection with which the publications are cited. Moreover, all definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The terms “can” and “may” are used interchangeably in the present disclosure, and indicate that the referred to element, component, structure, function, functionality, objective, advantage, operation, step, process, apparatus, system, device, result, or clarification, has the ability to be used, included, or produced, or otherwise stand for the proposition indicated in the statement for which the term is used (or referred to) for a particular embodiment(s).

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method of enriching a sample for one or more non-dominant Salmonella serotypes, the method comprising:
(a) obtaining a sample comprising (i) one or more dominant Salmonella serotypes; and
(ii) one or more non-dominant Salmonella serotypes;
(b) identifying the serotype of the one or more dominant Salmonella serotypes and of the one of more non-dominant Salmonella serotypes;
(c) contacting the sample with an antibody against one of the dominant Salmonella serotypes and allowing sufficient time for the antibody to bind;
(d) removing the antibody with the bound Salmonella serotype from the sample; and
(e) collecting the remaining sample and identifying the serotype of the remaining Salmonella serotypes.

2. The method of claim 1, wherein, before step (e), steps (c) and (d) are repeated for each of the dominant Salmonella serotypes in the sample.

3. The method of claim 1, wherein in step (c) the sample is contacted with an antibody against each of the dominant Salmonella serotypes simultaneously.

4. The method of claim 1, wherein one dominant Salmonella serotype is present, which accounts for at least 80%, at least 85%, at least 90%, at least 95%, or at least 98% of serotypes in the sample.

5. The method of claim 1, wherein one dominant Salmonella serotype is present, which accounts for at least 99% of serotypes in the sample, optionally where the dominant Salmonella serotype accounts for at least 99.9% of the serotypes in the sample.

6. (canceled)

7. The method of claim 1, wherein two or more dominant Salmonella serotypes are present, each of which accounts for at least 10%, a least 20%, at least 30%, at least 40%, or at least 50% of the serotypes in the sample.

8. The method of claim 1, wherein two or more dominant Salmonella serotypes are present, which cumulatively account for at least 80%, at least 85%, at least 90%, at least 95%, or at least 98% of serotypes in the sample.

9. The method of claim 1, wherein two or more dominant Salmonella serotypes are present, which cumulatively account for at least 99% of serotypes in the sample, optionally where the one or more dominant Salmonella serotypes account for at least 99.9% of the serotypes in the sample.

10. (canceled)

11. The method of claim 1, wherein one non-dominant Salmonella serotype is present, which accounts for less than 1% of serotypes in the sample, optionally wherein the non-dominant Salmonella serotype accounts for less than 0.1% of serotypes in the sample.

12. (canceled)

13. The method of claim 1, wherein two or more non-dominant Salmonella serotypes are present, each of which accounts for less than 1% of serotypes in the sample, optionally wherein each of which accounts for less than 0.1% of the serotypes in the sample.

14. (canceled)

15. The method of claim 1, wherein the dominant Salmonella serotype is not associated with human disease.

16. The method of claim 1, wherein the non-dominant Salmonella serotype is associated with human disease.

17. The method of claim 1, wherein the serotype identification in step (b) and/or (e) is carried out using CRISPR-SeroSeq or metagenomics.

18. The method of claim 1, wherein the antibody is an immune serum, a purified polyclonal antibody, a purified monoclonal antibody, an aptamer or another binder specific for the serogroup or serotype of the dominant serotypes.

19. The method of claim 1, wherein the antibody causes agglutination of the dominant serotype(s).

20. The method of claim 1, wherein the antibody is attached to a bead, optionally wherein the bead is made of latex, polymethyl methacrylate (PMMA), or polystyrene, or wherein the bead is magnetic.

21.-22. (canceled)

23. The method of claim 1, wherein the antibody is present on a membrane or filter.

24. The method of claim 1, wherein the antibody is present on the capture region of a microfluidic device, optionally wherein the microfluidic device is a ferrofluidic device.

25. The method of claim 1, wherein the sample is taken from a product intended for human or animal consumption.

26. The method of claim 1, wherein the sample is taken from an animal intended for human or animal consumption.

27. The method of claim 1, wherein the sample is taken from the environment of an animal intended for human or animal consumption.

28. The method of claim 1, wherein the sample is a pre-harvest sample.

29. The method of claim 1, wherein the sample is a post-harvest sample.
